(19) United States
(12) Patent Application Publication
(10) Pub. No.: US 2018/0078684 A1
(43) Pub. Date: Mar. 22, 2018

(54) CROSS-SHAPED DOUBLE SLEEVE DRAINAGE TUBE

(71) Applicant: GUANGZHOU SUN-SHING BIOTECH CO., LTD., Guangzhou, Guangdong (CN)

(72) Inventors: Yun PENG, Guangzhou, Guangdong (CN); Xi Feng YANG, Guangzhou, Guangdong (CN); Chen Guang ZENG, Guangzhou, Guangdong (CN)

(21) Appl. No.: 15/564,119

(22) PCT Filed: Jul. 30, 2015
(86) PCT No.: PCT/CN2015/085527

§ 371 Date: Oct. 3, 2017

(30) Foreign Application Priority Data
Apr. 9, 2015 (CN) 201510166764.2

(51) Int. Cl. A61M 1/00

(52) U.S. Cl. CPC A61M 1/008; A61M 1/0086; A61M 25/0043; A61M 2205/3344; A61M 1/0084

(57) ABSTRACT

The present invention discloses a cross-shaped double sleeve drainage tube, including an inner draining tube, a connecting member, an external sleeve tube, an inner guiding tube, a support rack, a block member, and a pressure sensor. A left end of the inner draining tube is formed in an opening, and a right end of the inner draining tube is sealed. A tube wall at the right end of the inner draining tube is provided with an inner draining hole. The connecting member combines the inner draining tube in the tunnel of the inner guiding tube. The left and right ends of the external guiding tube are formed in an opening, respectively. The tube wall at the right end of the external guiding tube is provided with a plurality of draining grooves. The inner guiding tube is installed in the tunnel of the external guiding tube through the support rack. The left and right ends of the inner guiding tube are formed in an opening, respectively, with the left end thereof connected with the tube wall of the external guiding tube. The tube wall of the inner guiding tube is provided with a pressure monitoring tunnel, an infusing tunnel, and an external draining hole. The block member includes a block head disposed in the right end opening of the inner guiding tube to block the opening and a block body to block the right end of the external guiding tube. A gap is formed between the left face of the block body and the right face of the external guiding tube. The pressure sensor is connected to the pressure monitoring tunnel.

### BACKGROUND OF THE INVENTION

### 1. Field of the Invention

The present invention relates to negative pressure drainage tubes, and more particularly, to a cross-shaped double sleeve drainage tube, which effectively prevents the draining holes from being blocked and monitors the instantaneous negative pressure strength of the wound surface, so as to prevent the tissue and organs in the body from being damaged due to an overpowered negative pressure.

### 2. Description of the Related Art

During a surgical operation treatment, blood, pus, tissue fluid, and excretion possibly accumulate at the lesion or wound. If the fluid and slough fragments are not immediately removed, the healing of the wound surface of the surgical operation will be affected, and further degeneration of the wound surface might occur in a worse case. Presently effectively solution applies a negative pressure draining technique to discharge the fluid and slough fragments. During the surgical operation, a negative pressure draining tube is inserted into the wound surface, so as to draining out the accumulate fluid through the negative pressure draining tube.

However, generally, tissue residual (blood clot or tissue to be removed) still exist at the lesion or the wound. During the draining process, the draining hole at the bottom end of the negative pressure draining tube is easily blocked by tissue residual. As a result, the negative pressure draining tube is unable to work properly, thereby affecting the normal execution of the surgical operation.

### SUMMARY OF THE INVENTION

For improving the issues above, the present invention provides a cross-shaped double sleeve drainage tube, which effectively prevents the draining holes from being blocked and monitors the instantaneous negative pressure strength of the wound surface, so as to prevent the tissue and organs in the body from being damaged due to an overpowered negative pressure. The negative pressure draining tube comprises an inner draining tube, an external sleeve tube, and a connecting member combining the inner draining tube in a tunnel of the external sleeve tube. A left end of the inner draining tube is formed in an opening, with a right end of the inner draining tube sealed. A tube wall of the right end of the inner draining tube is provided with at least an inner draining hole. A tube wall of the right end of the external sleeve tube is provided with at least one draining groove.

To further achieve the present invention, all the draining grooves are disposed in parallel. Direction of the length of draining grooves are arranged in alignment with the direction of the length of the external sleeve tube.

To further achieve the present invention, a right end of each draining groove extends rightward to the right end of the external sleeve tube.

To further achieve the present invention, a pressure sensor is further included. A tube wall of the inner draining tube or the external sleeve tube is provided with a pressure monitoring tunnel connected with the pressure sensor.

To further achieve the present invention, an opening at the right end of the pressure monitoring tunnel extends rightward to the right end of the inner draining tube or the external sleeve tube.

To further achieve the present invention, an opening of the right end of the pressure monitoring tunnel extends rightward to the right end of the inner draining tube or the external sleeve tube.

To further achieve the present invention, a tube wall of the inner draining tube or external sleeve tube is provided with an infusing tunnel connected with an external infusion bag.

To further achieve the present invention, an opening of the right end of the infusing tunnel extends rightward to the right end of the inner draining tube or the external sleeve tube.

To further achieve the present invention, the external sleeve tube includes an external guiding tube, an inner guiding tube, and a plurality of support racks. A left end of the external guiding tube is formed in an opening, with a right end of the external guiding tube sealed. A tube wall of the external guiding tube is provided with the draining groove. The inner guiding tube is installed in a tunnel of the external guiding tube through the support racks. A tube wall of the inner guiding tube is provided with external draining holes.

To further achieve the present invention, the external sleeve tube includes an external guiding tube, an inner guiding tube, a plurality of support racks, and a block member. A left end and a right end of the external guiding tubes are formed in an opening, respectively. A tube wall of the external tube is provided with the draining groove. The inner guiding tube is installed in a tunnel of the external guiding tube through the support racks. A left end of the inner guiding tube extends leftward to an inner wall of the external guiding tube. A tube wall of the inner guiding tube is provided with at least one external draining hole. The block member includes a block head and a block body. The block head is allowed to be inserted into the opening at the right end of the inner guiding tube to block the opening. The block member is disposed at the right end of the external guiding tube.

To further achieve the present invention, the connecting member includes a male connector and a female connector that are correspondingly connected and sealed. The male connector is fixedly connected with the external sleeve tube, and the female connector is fixedly connected with the inner draining tube. Through the combination of the male connector and the female connector, the inner draining tube is assembled in the tunnel of the external sleeve tube.

The present invention achieves following advantages:

1. Regarding the present invention, a plurality of draining grooves are disposed on the tube wall of the right end of the external sleeve tube. The relatively longer length of the draining grooves effectively prevents the draining holes of the external sleeve tube from being adhered by membrane tissues or larger slough tissue in the human body, so as to assure that the waste fluid is properly discharged.

2. Regarding the present invention, the tube body of the external sleeve tube comprises a structure including the external guiding tube, the inner guiding tube, and the support racks. By blocking the opening at the right end of the inner guiding tube, the tube body is compression resisting, and the discharge of the waste fluid is assured.

3. Regarding the present invention, the inner draining tube or the external sleeve tube of the present invention is provided with a pressure monitoring tunnel. The pressure monitoring tunnel is connected to a pressure sensor. During the draining process under a negative pressure, the pressure sensor is applied to immediately monitoring the actual strength of the negative pressure at the wound location of the surgical operation, so as to prevent the tissue and organs in the body from being damaged due to an overpowered negative pressure at the wound location (A certain difference exists between the actual negative pressure strength at the wound surface and the pressure output by the pressure pump.).

4. The inner draining tube or the external sleeve tube is provided with an infusing tunnel. By infusing cleansing fluid or disinfectant to the wound location, no additional cleansing tube is needed to be inserted into the wound, efficiently facilitating the surgical operation for the doctor.

### BRIEF DESCRIPTION OF THE DRAWINGS

FIG. 1 is a structural schematic view illustrating the cross-shaped double sleeve drainage tube in accordance with the present invention.

FIG. 2 is a structural schematic view illustrating the inner draining tube in accordance with the present invention.

FIG. 3 is a structural schematic view illustrating the external sleeve tube in accordance with the present invention.

FIG. 4 is a sectional schematic view illustrating the external sleeve tube in accordance with the present invention.

### DETAILED DESCRIPTION OF THE INVENTION

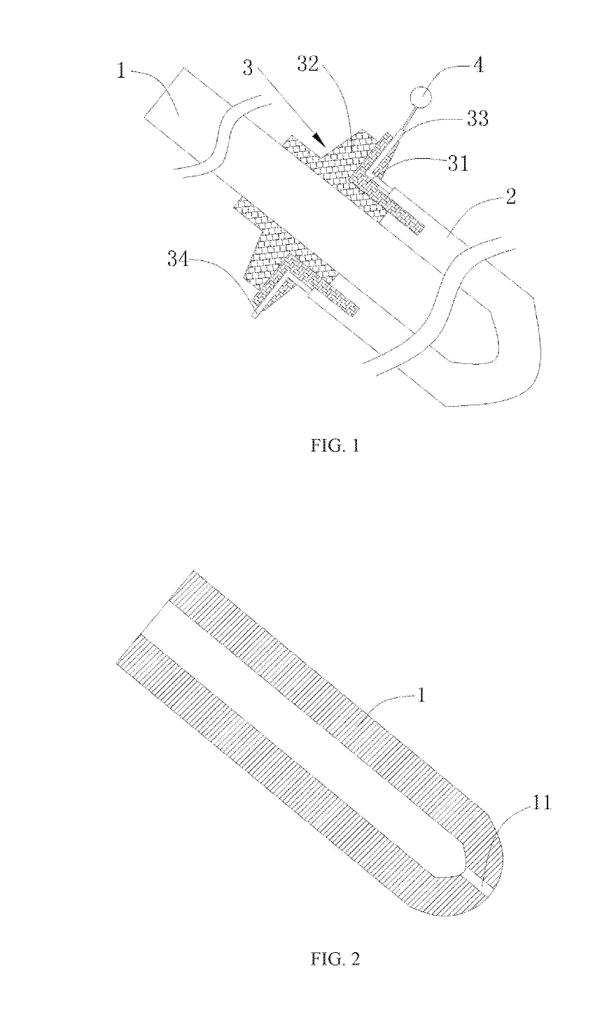

The present invention is explained in conjunction with the accompanying drawings as following, wherein the direction referred in the present invention is defined based on FIG. 1.

Referring to FIG. 1, a cross-shaped double sleeve drainage tube in accordance with the present invention comprises an inner draining tube 1, an external sleeve tube 2, a connecting member 3, and a pressure sensor 4. One end of the inner draining tube 1 is connected with an external negative pressure source, and the other end thereof is inserted into the tunnel of the external sleeve tube 2, so as to drain the waste fluid at the location of the wound surface of the surgical operation by use of the negative pressure. The external sleeve tube 2 is applied for preventing the membrane tissues or larger slough tissues in the body from blocking the inner draining tube 1. The connecting member 3 is applied for combining the inner draining tube 1 in the tunnel of the external sleeve tube 2. The pressure sensor 4 is applied for monitoring the actual strength of the negative pressure during draining from the wound surface of the surgical operation by use of the negative pressure.

Referring to FIG. 2, the inner draining tube 1 is formed of a silica gel material. The left end of the inner draining tube 1 is connected with an external negative pressure source, and the right end thereof is sealed. The tube wall of the right end of the inner draining tube 1 is provided with a plurality of draining holes 11 spaced with each other. The right end of the inner draining tube 1 is received in the tunnel of the external sleeve tube 2.

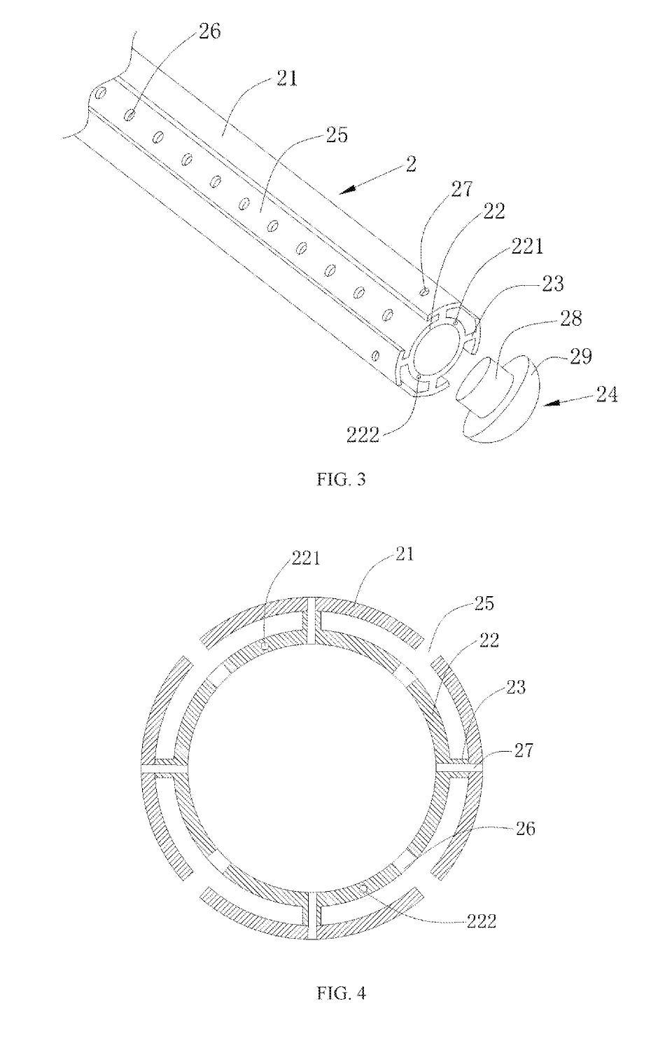

Referring to FIG. 3 and FIG. 4, the external sleeve tube 2 comprises an external guiding tube 21, an inner guiding tube 22, support racks 23, and a block member 24. The external guiding tube 21 is formed of a silica gel material, with the left end and the right end thereof formed in an opening, respectively. A plurality of draining grooves 25 formed in opening grooves are disposed along the tube wall of the right end of the external guiding tube 21, and all draining grooves 25 are disposed in parallel, with the direction of the length of draining grooves 25 arranged in alignment with the direction of the length of the external sleeve tube 21, with the right end of each draining groove 25 extending to an end of the external guiding tube 21. The inner guiding tube 22 is formed of a silica gel material, with the left end and the right end thereof formed in an opening, respectively. The inner guiding tube 22 is installed in the tunnel at the right end of the external guiding tube 21 through the support racks 23, and located at a section of the external guiding tube 21 that is provided with corresponding draining grooves 25. The left end of the inner guiding tube 22 is connected with the inner wall of the external guiding tube 21, so as to form a tunnel. The tube wall of the inner guiding tube 22 is provided with a plurality of external draining holes 26 passing therethrough at the locations in alignment with the corresponding draining grooves 25, respectively. Also, the tube wall of the inner guiding tube 22 is provided with a pressure monitoring tunnel 221 and an infusing tunnel 222. The direction of the length of the pressure monitoring tunnel 221 and the infusing tunnel 222 are arranged in alignment with the direction of the length of the inner guiding tube 22. The right end openings of the pressure monitoring tunnel 221 and the infusing tunnel 222 are disposed at the right end of the inner guiding tube 22, respectively. The support racks 23 are provided in an amount of four, wherein the direction of the length of the support racks 23 are identical to the direction of the length of the external guiding tube 21. A central draining hole 27 is disposed at the junction where the support rack 23 and the inner guiding tube 22 are connected with the external guiding tube 21. The block member 24 is disposed at the opening at the right end of the inner guiding tube 22. The block member 24 is formed of a silica gel material. The block member 24 includes a block head 28 and a block body 29. The block head 28 is allowed to be inserted in the opening at the right end of the inner guiding tube 22 to block the opening. The block body 29 is disposed at the opening of the right end of the external guiding tube 21, with a gap formed between the left face of the block body 29 and the right face of the external guiding tube 21.

The connecting member 3 includes a male connector 31 and a female connector 32 that are correspondingly connected and sealed. The male connector 31 is fixedly connected with the right end of the external sleeve tube 2. The male connector 31 is also provided with a pressure connector 33 connected with the pressure monitoring tunnel 221, and an infusion connector 34 connected with the infusing tunnel 222. The female connector 32 is fixedly connected to a middle section of the outer tube wall of the inner draining tube 1. With the combination of the male connector 31 and the female connector 32, the right end of the inner draining tube 1 is disposed in the tunnel of the external sleeve tube 2.

The pressure sensor 4 is connected with the pressure connector 33 of the connecting member 3. The pressure sensor 4 is able to immediately monitor the actual strength of the negative pressure at the location of the wound surface of the surgical operation, so as to prevent the tissue and organs in the body from being damaged due to an overpowered negative pressure at the location of the wound surface (A certain difference exists between the actual negative pressure strength at the wound surface and the pressure output by the pressure pump.). Meanwhile, blockage of the present invention is allowed to be identified according to the data monitored by the pressure sensor 4 (A dramatic rise of the data monitored by the pressure sensor 4 dramatically indicates a blockage occurring.).

During the draining process by use of the negative pressure, the connecting member 3 is first applied to efficiently combine the inner draining tube 1 and the external sleeve tube 2, and the right end of the inner draining tube 1 is disposed to be adjacent to the right end of the external sleeve tube 2 as close as possible. Then, the right end of the external sleeve tube 2 is inserted or implanted into the location of the wound surface of the surgical operation. By use of the external draining holes 26 and the central draining holes 27, the fluid at the location of the wound surface of the surgical operation is guided into the tunnel of the inner guiding tube 22 and finally drained out by the inner draining tube 1 by the use of the negative pressure. The pressure monitoring tunnel 221 is connected with the pressure sensor 4 at the same time, so as to immediately monitor the actual strength of the pressure at the location of the wound surface of the surgical operation. The infusing tunnel 222 is connected with an external infusion bottle or infusion bag, thereby cleansing and disinfecting the wound surface of the surgical operation.

Furthermore, the pressure monitoring tunnel 221 and the infusing tunnel 222 in accordance with the present invention are allowed to be disposed on the tube wall of the inner draining tube 1 as well. The female connector 32 of the connecting member 3 is further provided with the pressure connector 33 connected with the pressure monitoring tunnel 221, and the infusion connector 34 connected with the infusing tunnel 222.

Although preferred embodiments of the invention have been described above, the present invention is not limited to the aforementioned embodiments. Various partial or trivial structural modifications and enhancements may be made during the execution. If modifications or variations of the present invention are made without departing from the spirit and scope of the invention and belong to the same technical scope, such modifications and variations are included in the present invention.

1. A cross-shaped double sleeve drainage tube, comprising:
an inner draining tube;
an external sleeve tube; and
a connecting member combining the inner draining tube in a tunnel of the external sleeve tube;
a left end of the inner draining tube is formed in an opening, and a right end of the inner draining tube is sealed; a tube wall at the right end of the inner draining tube is provided with at least an inner draining hole,
wherein:
a tube wall of a right end of the external sleeve tube is provided with at least a draining groove.

2. The cross-shaped double sleeve drainage tube of claim 1, wherein: the draining grooves are disposed in parallel, and a length and a direction of the draining groove is identical to a length and a direction of the external sleeve tube.

3. The cross-shaped double sleeve drainage tube of claim 1, wherein: a right end of each draining groove extends rightward to a right end of the external sleeve tube.

4. The cross-shaped double sleeve drainage tube of claim 1, wherein: a pressure sensor is further included, a tube wall of the inner draining tube or the external sleeve tube is provided with a pressure monitoring tunnel connected with the pressure sensor.

5. The cross-shaped double sleeve drainage tube of claim 4, wherein: an opening at a right end of the pressure monitoring tunnel extends rightward to a right end of the inner draining tube or the right end of the external sleeve tube.

6. The cross-shaped double sleeve drainage tube of claim 1, wherein: the tube wall of the inner draining tube or the external sleeve tube is provided with an infusing tunnel connected with an external infusion bag.

7. The cross-shaped double sleeve drainage tube of claim 6, wherein: an opening at a right end of the infusing tunnel extends rightward to the right end of the inner draining tube or the external sleeve tube.

8. The cross-shaped double sleeve drainage tube of claim 1, wherein: the external sleeve tube comprises an external guiding tube, an inner guiding tube, and a plurality of support racks; a left end of the external guiding tube is formed in an opening, and a right end of the external guiding tube is sealed; a tube wall of the external guiding tube is provided with the draining groove; the inner guiding tube is installed in a tunnel of the external guiding tube through the support racks; a tube wall of the inner guiding tube is provided with an external draining hole.

9. The cross-shaped double sleeve drainage tube of claim 1, wherein: the external sleeve tube comprises an external guiding tube, an inner guiding tube, a plurality of support racks, and a block member; a left end and a right end of the external guiding tube are formed in an opening, respectively; a tube wall of the external guiding tube is provided with the draining groove; the inner guiding tube is installed in a tunnel of the external guiding tube through the support racks; a left end of the inner guiding tube extends leftward to an inner wall of the external guiding tube; a tube wall of the inner guiding tube is provided with at least an external draining hole; the block member includes a block head and a block body, the block head being disposed in the opening at the right end of the inner guiding tube to block the opening, the block body is disposed at the opening of the right end of the external guiding tube.

10. The cross-shaped double sleeve drainage tube of claim 1, wherein: the connecting member includes a male connector and a female connector are correspondingly connected and sealed with each other; the male connector is fixedly connected with the right end of the external sleeve tube, and the female connector is fixedly connected to the inner draining tube, such that the inner draining tube is assembled in the tunnel of the external sleeve tube through a combination of the male connector and the female connector.
